(19) United States
(12) Patent Application Publication
(10) Pub. No.: US 2024/0115307 A1
(43) Pub. Date: Apr. 11, 2024

(54) PROBE WITH DISTAL CUTTING ELECTRODE

(71) Applicant: Erbe Elektromedizin GmbH, Tuebingen (DE)

(72) Inventors: Marcus Adler, Burladingen (DE); Felix Karcher, Tuebingen (DE)

(73) Assignee: Erbe Elektromedizin GmbH, Tuebingen (DE)

(21) Appl. No.: 18/377,715

(22) Filed: Oct. 6, 2023

(30) Foreign Application Priority Data
Oct. 7, 2022 (EP) 22200337.8

(51) Int. Cl. A61B 18/14

(52) U.S. Cl. CPC A61B 18/14; A61B 2018/00601

(57) ABSTRACT

A probe having a cutting electrode at its distal end, which is formed on an end piece of probe. From the end piece a wire extends over the entire length of probe therethrough. The wire can serve for current supply to the end piece. The cutting electrode can include a junction site from which electrode surfaces extend away in at least three directions.

### CROSS-REFERENCE TO RELATED APPLICATION

This application claims priority to European Patent Application No. 22200337.8, filed Oct. 7, 2022, the entirety of which is incorporated by reference herein.

The invention refers to a probe, particularly for HF-ablation of biological tissue.

Probes for HF-ablation serve, for example, the devitalization of tissue sections, e.g. the devitalization of tumors or the like embedded in tissue (e.g. lung or liver).

EP 3 788 974 A1, EP 1 181 896 A1 and US 2005/0010201 A1 respectively disclose an ablation probe of the indicated configuration having electrodes held on a hose-like body that are to be connected with an output of an electrical generator. The electrodes serve to heat the tissue, due by applying current, if the ablation probes are pierced into tissue. In order that tissue being in direct contact with the electrodes does not get high-ohmic due to drying, but remains moist and thus low-ohmic, the electrodes are cooled. For this purpose a fluid capillary is arranged in the lumen of the hose of such an ablation electrode, via which cooling fluid exits and cools the electrodes.

The piercing of a probe in biological tissue is a critical task, particularly in the case of tumor treatment. Particularly entering encapsulated tumors or upstream located structures being difficult to pass shall be made possible. In addition, a controlled piercing without high effort shall be made possible, because this could result in penetrating through the tumor. Such a penetration would result in an injury of downstream structures or the carryover of tumor cells. It shall also be avoided that tumor cells can be carried into healthy tissue in case of penetration of the tumor.

A biopsy instrument is known from U.S. Pat. No. 6,454,727 B1 that is provided with a bracket-shaped electrode on the outside at its distal end. The electrode is connected with an electrical current supply and cuts into tissue, if it is activated.

Starting therefrom it is the object of the invention to provide an improved probe.

This object is solved by means of the probe according to claim 1:

The probe comprises a flexible hose on which two or more electrodes can be arranged with distance to one another in axial direction of the hose. Preferably such electrodes can thereby extend around the entire circumference of the hose as well as over an axial length (to be measured between its distal and its proximal end). The electrodes are preferably flexible so that they can follow tight lateral curvatures. For this purpose they can be configured in the type of helical springs.

The electrodes are connected to electrical lines that extend from the respective electrode up to the proximal end of the hose and can there be connected by a suitable connector with a supplying generator. The supplying generator is preferably configured to provide a high frequency alternating voltage and thus to output high frequency current.

The hose encloses a lumen that extends from its proximal end up to its distal end and is closed there. At the distal end an end piece is provided that is rigidly connected with the hose and can close the lumen thereof. Inside the lumen a fluid supply line is arranged that extends from the proximal end of the hose in distal direction up to the electrodes. At the proximal end the fluid supply line can be provided with a suitable connection device in order to be connected with a cryofluid source that is, for example, arranged in the supplying apparatus. The supplying apparatus can thus include the cryofluid source and the generator for supplying current to the electrodes in one apparatus. Separated configurations are also possible. Such separate apparatus can be connected by a suitable interface, e.g. a BUS, to form an apparatus system.

The end piece for closing the hose supporting the electrodes can consist of an insulating material, e.g. plastic or ceramic, or also of metal. Particularly, it can also consist of a combination of an electrically insulating material and an electrically conductive material. Electrically conductive areas of the end piece can be used as electrodes, for example, in order to support piercing of the probe into biological tissue. Particularly the end piece comprises an extension serving as cutting electrode that is enclosed by an insulator leaving a strip-shaped distal end surface uncovered. Preferably the uncovered end surface projects only slightly or does not project from the insulator. The so formed cutting electrode supports the piercing of the probe into biological tissue and keeps a lateral damage of tissue low. Particularly it is thereby possible to penetrate through tissue without thereby negatively influencing the subsequent ablation. Also, the formation of a too large coagulation zone around the penetration position can be avoided, which would result in an increase of the impedance of the tissue to be abladed. Particularly, it is thereby possible to penetrate through malign tissue or a rigid tissue structure without thereby carrying over malign tissue into healthy tissue or due to the effort during penetration unintentionally injure healthy tissue.

The distal cutting electrode of the probe according to the invention in addition reduces the mechanical resistance during penetration into tissue, particularly into compact tumor tissue. The cutting electrode can devitalize and cut tissue located in front of it during piercing, so that it can easily enter into the tissue. Because in doing so, remarkably lower thrust power has to be applied compared with exclusively mechanical penetration without a cutting electrode, it is now possible to configure the probes particularly flexible. This facilitates the further downsizing of the ablation electrode, i.e. probes with particularly small diameter can be realized. In addition, very small bending radii can be achieved, which increases the spectrum of application and treatment possibilities.

Preferably the end piece is connected to a tension-resistant wire that extends from the end piece through the lumen of the probe up to the proximal end thereof and is anchored there. The wire serves to support tensile forces during removal of the probe from a patient, particularly from its tissue. The wire relieves the hose from tensile forces so that the probe can be reliably retrieved in any case, also in case of treatment problems, e.g. as a result of sticking of the ablation electrodes in the tissue. The traction wire preferably comprises a high restoring force after deformation induced by an external force, e.g. by bending the access system.

The end piece of the probe can consist of an electrically conductive ceramic or of a metal, e.g. stainless steel or hard metal. The end piece can be provided with a coating, e.g. a silver coating, entirely or also only in the area of the extension or also only on the end surface serving as cutting electrode. In doing so, the cutting characteristics can be positively influenced.

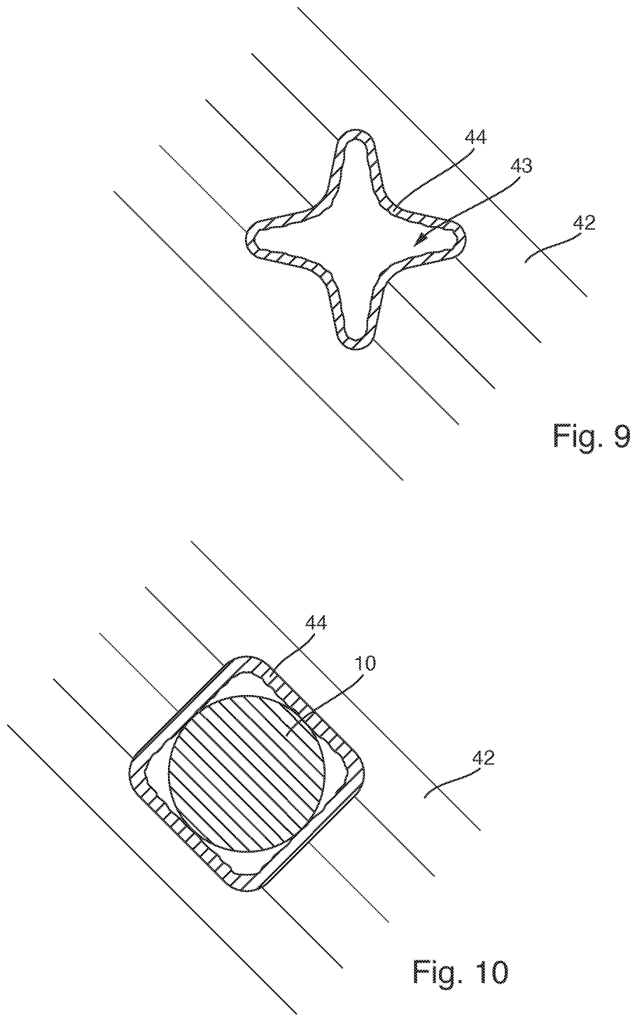

The distal end surface of the probe can comprise a junction site from which multiple sections extend. The junction site can be arranged axially centered as shown in FIGS. 7 and 8 (references 37, 38). The strip-shaped areas having cutting effect open a channel in the tissue in this manner through which the probe can be moved easily and also without carryover of tissue.

In the probe according to the invention the sections can be arranged following a pyramid shape, a cone shape, an ellipsoid shape or a spherical shape. The tissue channel opened by cutting by the cutting electrode can be easily widened in this manner. This applies particularly, if the extension of the probe is configured having three or multiple vanes. For this purpose the extension can comprise multiple vanes that preferably extend radially away from a center axis.

The end piece of the probe can consist of an insulator and a metal body that is uncovered at the distal end surface. It is, however, also possible that the insulator is formed by a coating of the metal body. Thereby the coating can be an electrically non-conductive coating of ceramic or plastic that covers parts of the distal face of the end piece and leaves sections therebetween uncovered that form the cutting electrode.

The end piece of the probe can be configured to be connectable to a cutting current source. In addition, at or on the hose one or more electrodes can be arranged. At least one of these electrodes can be connectable to an ablation voltage source and can serve in this manner as return electrode (neutral electrode) for the current originating from the cutting electrode. It is, however, also possible to use an extensive electrode attached to the patient at another location as neutral electrode.

A method for inserting a probe in a tumor or other tissue is part of the invention during which the probe is pierced into tissue comprising a region that requires treatment, e.g. a tumor. During piercing the end piece is applied with a cutting voltage. This simplifies penetration of the tissue, particularly of more rigid tissue. Thereby the probe can be positioned sensitively without having to fear carryover of tissue along the formed penetration channel.

After having carried out the treatment, the probe is retracted through the penetration channel after ablation of the tumor. For avoiding carryover of tumor cells it can be provided that the tip electrode and/or the ablation electrode(s) are supplied with current in a special mode. In doing so, the tissue surrounding the penetration channel can be coagulated after the ablation.

Further details of advantageous embodiments of the invention are subject of dependent claims. In the drawing an embodiment of the invention is illustrated. The drawing shows:

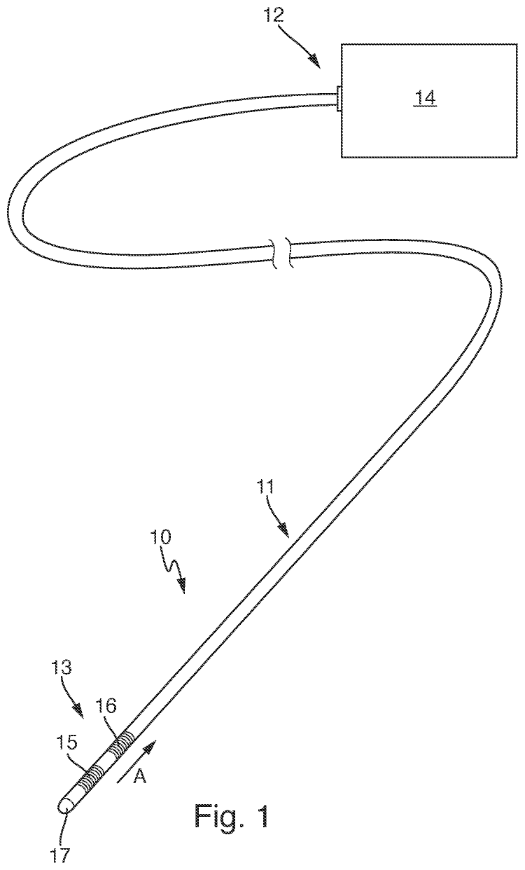

FIG. 1 the probe according to the invention connected to a supplying apparatus in a perspective principle illustration,

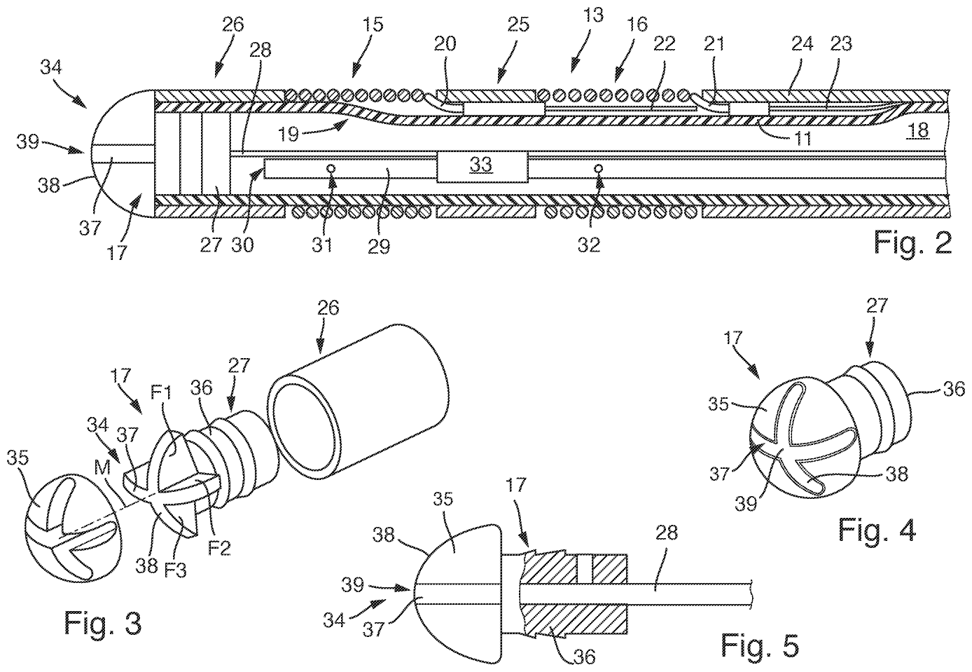

FIG. 2 the distal end of the probe cut in longitudinal direction with view into its lumen,

FIG. 3 an end piece for the probe according to FIG. 1 or 2 in a perspective explosion illustration,

FIG. 4 the end piece according to FIG. 3 in partly perspective illustration,

FIG. 5 the end piece according to FIG. 3 with an insulator in a cut side view,

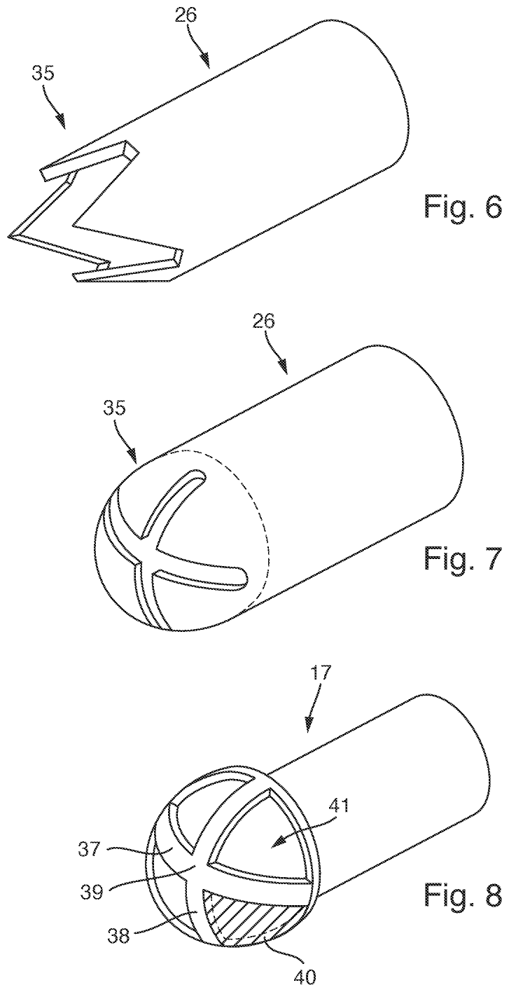

FIGS. 6 and 7 different variants of the insulator in perspective illustration,

FIG. 8 an end piece having a cutting electrode and pockets for location of insulating material in a perspective illustration,

FIG. 9 biological tissue with pierced opening without probe and

FIG. 10 biological tissue with pierced opening with probe.

FIG. 1 illustrates a probe 10 as it can be used for the endoscopic use, e.g. for devitalization of tumors or other tissue (e.g. lung tumors), but also for other purposes. The probe 10 comprises an elongated flexible hose 11 that extends from a proximal end 12 up to a distal end 13 of the probe 10. At the proximal end 12 one or more plugs or another suitable connection device is provided in order to connect the probe 10 with the supplying apparatus 14.

The hose 11 is preferably a flexible plastic hose that is made of a suitable biocompatible plastic, e.g. PE/PET, PEEK or similar. While its length can have multiple meters, the diameter is preferably in a range of 1 mm to 3 mm. Other dimensions are possible and expedient depending on the application.

The distal end 13 of the probe 10 can comprise one or multiple electrodes 15, 16 that are distanced from one another in axial direction A. The electrode 15 and at least one additional electrode 16, which can be provided if necessary, can serve for tissue ablation.

The hose 11 is in addition provided with an end piece 17 at its distal end, which distally closes its inner lumen 18 apparent from FIG. 2.

The hose 11 surrounds this lumen 18 and is thereby preferably configured without gaps and without interruptions. The electrodes 15, 16 are arranged on the hose 11 in an area of a trough 19 formed in the hose 11. Connections 20, 21 of the electrodes 15, 16 are located in trough 19. From the connections 20, 21 electrical lines 22, 23 extend along the hose 11 on the outside in proximal direction up to end 12. There they are connected to a plug or another connection device for apparatus 14.

The lines 22, 23 extend through a gap between the hose 11 and a cover hose 24 that is preferably formed from a very flexible slidable plastic. Between the electrodes 15, 16 a sleeve-shaped insulator 25 can be arranged. In addition, another insulator can be arranged between the distal electrode 15 and the end piece 17.

The end piece 17 is preferably rounded at its distal outer surface. For example, it can be configured hemispherically. From the hemispheric head a shank 27 extends into the lumen, whereby the shank 27 is secured to the hose 11 in a form-fit manner and/or by adhesive or other suitable attachment means. In addition, the end piece 17 is preferably connected with a wire 28 that extends originating from the end piece up to the force transmitting part of the probe 10, which is, for example, arranged at the proximal end 12 of the probe 10, in order to reliably transmit tensile forces from the proximal end onto the distal end 13. The force transmitting part of the probe 10 can also be arranged in the course of the length of the probe 10.

Inside lumen 18 a fluid supply line 29 can be arranged that can be configured as thin plastic hose, but if required also as metal capillary. The fluid supply line 29 serves to release cooling fluid at its distal end 30 or one or more lateral openings 31, 32. The distal end of the fluid supply line can be accordingly open or can be closed, e.g. by adhesive or by a stopper. The fluid cools the electrodes 15, 16 and then flows through lumen 18 back to apparatus 14. There or at the proximal end 12 of probe 10 it can be released into the environment or can also be captured and recycled.

In order to guarantee the sufficiently accurate axial relative position of the openings 31, 32 relative to the electrodes 15, 16, the fluid supply line 29 can be axially fixated at its distal end. For this purpose it can be connected to the wire 28, e.g. by a shrinking hose 33, a clamp or the like. The wire can be made of a spring steel or another particularly tension-resistant material, which preferably comprises a high restoring tendency after deformation, such as nitinol. Also, the use of a plastic wire is possible. Alternatively, the traction wire 28 can be configured in a hollow manner and serve as fluid supply line 29. As illustrates FIG. 5, the end piece 17 is connected to the traction wire 28 in a tension-resistant manner, e.g. welded, press-connected and/or adhesively connected. Particularly the connection is electrically conductive.

The end piece 17 is separately apparent from FIGS. 3 and 4. It forms a cutting electrode 34 and for this purpose can be completely made of an electrically conductive material, e.g. as metal body 36, as hard metal body from tungsten carbide or also from electrically conductive ceramic. The end piece 17 can comprise multiple (at least three) vanes F1, F2, F3 that extend originating from a center axis M, preferably radially outwardly and on which the cutting electrode 34 is formed.

As illustrated in FIGS. 3 and 4, an insulator 35 can be assigned to the end piece 17, wherein the insulator covers and thus insulates areas of the end piece 17 to the outward that do not serve as cutting electrode. The insulator can hold the vanes F1, F2, F3.

At its rounded end surface (e.g. at its vanes F1, F2, F3) the metal body 36 comprises electrode surfaces 37, 38 that extend through respective openings of the insulator 35 and form the cutting electrode 34. The latter can also slightly project distally beyond the insulator 35. For example, the metal body 36 can thereby be cross-shaped at its distal end, as illustrated in FIG. 3. In this case the electrode surfaces 37, 38 branch off a central junction site 39 radially, however, following the approximately spherical curvature of the distal end of the closure piece 17. An electrode structure having four vanes results in which the electrode sections extending from the junction site 39 include in pairs angles of 90° with each other. The electrode surfaces 37, 38 thus branch off the junction site 39 in four different directions. However, also electrode structures having three vanes with angles of 120° between the electrode surfaces can be provided so that the electrode surfaces branch off the junction site 39 (radially) outwardly in three directions. Also, structures having five or more vanes can be selected, whereby the electrode surfaces then branch off the junction site 39 (radially) outwardly in five or more directions.

Preferably the insulator 35 is made of a plastic, particularly a plastic resistant against high temperatures and resistant against creeping currents, or ceramic. It comprises an opening that is adapted to the shape of the electrode surfaces 37, 38 and the edge of which adjoins the electrode surfaces. The insulator 35 can be configured as separate part, as illustrated in FIG. 3 or can be alternatively also configured monolithically together with insulator 26, so that they form a single common part.

Such variants of the insulators 26, 35 are illustrated in FIGS. 6 and 7. FIG. 6 illustrates the insulator 35 that is depicted as crown-like extension of the sleeve-shaped insulator 26. After application of the insulator 26 on the distal end of hose 11, the four parts of the insulator 35 illustrated in FIG. 6 can be deformed radially inwardly, so that the insulator 35 takes the shape according to FIG. 7. The body enclosing the insulators 26, 35 can, however, also have the shape illustrated in FIG. 7 before its attachment to the probe and can be placed in this shape on the distal end of hose 11 after insertion of the end piece 17 in the end of hose 11.

It is in addition possible to configure the insulator 35 by insulating inlays or coatings 40 that are arranged in respectively shaped depressions or pockets 41 of end piece 17. FIG. 8 illustrates such an end piece 17 in which these pockets 41 are formed between the metallic bare electrode surfaces 37, 38. The inlays or coatings can consist of plastic, ceramic, glass or another electrical non-conductor. In turn the electrode surfaces 37, 38 can form an electrode having four vanes, as illustrated, but alternatively also an electrode having three, five or more vanes. The three or four-vane variant is, however, considered to be the best embodiment.

The traction wire 28 supporting the tensile forces can be provided with an electrical connection device at the proximal end 12 in order to be supplied with voltage and current from apparatus 14. In doing so, the electrode surfaces 37, 38 of metal inlay 36 become effective as cutting electrode, e.g. during penetration of the probe 10 in biological tissue.

The probe 10 described so far operates as follows:

The probe 10 is inserted solely or via a respective access instrument, e.g. an endoscope or in case of an ablation of a lung tumor a bronchoscope, in the bronchial tree of the patient. Then probe 10 is further moved in distal direction out of the endoscope toward the tissue that requires treatment, e.g. the lung tumor, and pierced therein. For this purpose the cutting electrode formed by the electrode surfaces 37, 38 is supplied with electrical voltage, the amount of which is sufficient for achieving a tissue cut. The electrode surfaces 37, 38 first create a cross-shaped cut 43 having a coagulation boundary 44 in the biological tissue 42, as schematically illustrated in FIG. 9. During forward movement of probe 10 the cross-shaped cut is widened by probe 10, whereby the probe slides along the coagulated surfaces of the coagulation boundary 44. Due to the smooth outer shape of the probe and the electrical creation of the cut 43, only a small thrust force is necessary for piercing the probe 10 into the tissue 42. Therefore, probe 10 can be designed very slim and flexible. In addition, the coagulation of tissue 42 and the formation of the coagulation boundary 44 avoids a carryover of tissue in piercing direction during penetration of tumors.

After piercing of probe 10, electrodes 15 and 16 are applied with treatment voltage and provided with treatment current. Concurrently or shortly before/afterwards the fluid supply line 29 is supplied with cooling fluid, so that it exits from the openings 31, 32. The cooling fluid, e.g. carbon dioxide, is provided in the fluid supply line under a high pressure of some 10 bar (e.g. 65 bar) and passes through the openings 31, 32 into lumen 18 in which a lower pressure of at most a few bar is present. The openings 31, 32 thereby serve as throttle openings, whereby cold is produced due to the Joule-Thomson-effect. Due to the fixed registration, i.e. axial alignment of openings 31, 32 in relation to the electrodes 15, 16, the cooling effect is particularly approximately created centrally relative to the electrodes 15, 16, so that a largely uniform cooling of each electrode 15, 16 is guaranteed. In doing so, a too extensive heating of electrodes 15, 16 and thus a drying of the abutting tissue is avoided, even if the diameter of probe 10 is reduced to very low values of, for example 2.3 mm or less, which results in high current densities at the electrodes 15, 16. By means of the concept according to the invention, however, an improvement and homogenization of the electrode cooling is effected, whereby the quality of the ablation treatment is increased.

It has to be noted that probe 10 cannot only be provided as ablation probe, as described above, but without electrodes 15, 16 also as cryoprobe for example, or without inner cooling also as electrical needle for penetrating into the tissue 42, i.e. for creation of a channel provided with a coagulation boundary 43.

A probe 10 according to the invention comprises a cutting electrode 34 at its distal end, which is formed on an end piece 17 of probe 10. From the end piece 17 a wire 28 extends over the entire length of probe 10 therethrough. The wire 28 can serve for current supply to the end piece 17. According to the invention, cutting electrode 34 comprises a junction site 39 from which electrode surfaces 37, 38 extend away in at least three directions.

1. A flexible hose for a surgical probe comprising:
an electrode; and
an electrically conductive end piece at a distal end of the hose,
the end piece having a cutting electrode, and
an insulator partially covering the cutting electrode such that the insulator forms two strip-shaped distal electrode surfaces exposed at the distal end of the hose.

2. The flexible hose according to claim 1, wherein the cutting electrode is metal.

3. The flexible hose according to claim 1, wherein the distal electrode surfaces comprise a junction site from which they extend away in at least three different directions.

4. The flexible hose according to claim 3, wherein the distal electrode surfaces are arranged following a pyramid shape, a cone shape, an ellipsoid shape or a ball shape.

5. The flexible hose according to claim 1, wherein the end piece is configured having three or more vanes.

6. The flexible hose according to claim 5, wherein the end piece comprises multiple vanes that extend away from a center axis.

7. The flexible hose according to claim 1, wherein the end piece consists of an insulator (35) and a metal body.

8. The flexible hose according to claim 1, wherein the insulator is a coating over the metal body.

9. The flexible hose according to claim 1, further comprising a wire that extends through a lumen and wherein the end piece is connected to the wire.

10. The flexible hose according to claim 1, wherein the end piece is configured to be connectable with a cutting current source.

11. The flexible hose according to claim 1, wherein the electrode is arranged on the hose.

12. The flexible hose according to claim 1, wherein the electrode can be connected to an ablation voltage source.

13. The flexible hose according to claim 12, wherein the electrode can be connected for receiving a current originating from the cutting electrode.

14. A method for using a probe for surgical procedures comprising the steps of:
inserting a probe into tissue that comprises an area requiring treatment, the probe comprising;
an electrode; and
an electrically conductive end piece at a distal end of the hose,
the end piece having a cutting electrode, and
an insulator partially covering the cutting electrode such that the insulator forms two strip-shaped distal electrode surfaces exposed at the distal end of the hose piercing into the tumor and thereby positioning electrode in the tissue requiring treatment until it reaches healthy tissue.
